(19) United States
(12) Patent Application Publication
(10) Pub. No.: US 2025/0325462 A1
(43) Pub. Date: Oct. 23, 2025

(54) COACERVATE CLEANSING COMPOSITION WITH MINERAL PARTICLE

(71) Applicant: L'OREAL, PARIS (FR)

(72) Inventor: Changlong CHEN, New Brunswick, NJ (US)

(73) Assignee: L'OREAL, PARIS (FR)

(21) Appl. No.: 18/640,421

(22) Filed: Apr. 19, 2024

(51) Int. Cl. A61K 8/44; A61K 8/04; A61K 8/27; A61K 8/29; A61K 8/46; A61K 8/60; A61K 8/73; A61K 8/88; A61Q 17/04; A61Q 19/10

(52) U.S. Cl. CPC A61K 8/442; A61K 8/04; A61K 8/27; A61K 8/29; A61K 8/466; A61K 8/602; A61K 8/736; A61K 8/88; A61Q 17/04; A61Q 19/10; A61K 2800/30

(57) ABSTRACT

The disclosure relates to compositions comprising (a) at least one amphoteric surfactant, (b) at least one glycolipid, (c) at least one cationic polymer from about 0.05% to about 2%, (d) at least one mineral particle, and (e) at least one cosmetically acceptable solvent wherein the cleansing system is essentially free of phenoxyethanol, and wherein the pH of the composition is between about 4 and about 8, and wherein the weight ratio of a to b is about 0.4 to about 4, and wherein the total concentration of a and b combined is about 10% to about 20% of the total weight of the composition. The compositions can be used as rinse-off compositions for cleansing skin and depositing mineral particles.

### TECHNICAL FIELD

The present disclosure is directed to a skin care composition, in particular, a coacervate cleansing composition containing mineral particle.

### BACKGROUND

Current skincare routines usually require multiple steps and multiple products to achieve desired skincare results. Typical skincare routines usually include a skin cleansing step followed by another product to impart various skin benefits such as hydration, brightening, or UV protection. Common skin cleansing products usually rely on surfactants to provide cleansing and do not deposit skincare actives onto the skin.

There is a need in the market for skin cleansing compositions to provide enhanced deposition of skincare actives, in particular mineral particles. As such, consumers desire new and improved skin cleansing compositions that can cleanse the skin and also deposit mineral particles for brightening or sun protection purposes.

### SUMMARY

In various embodiments, provided is a coacervate cleansing system, comprising:
(a) at least one amphoteric surfactant;
(b) at least one glycolipid;
(c) at least one cationic polymer from about 0.05% to about 2%;
(d) at least one mineral particle; and
(e) at least one cosmetically acceptable solvent;
   wherein the cleansing system is essentially free of phenoxyethanol and wherein the pH of the composition is between 4 and 8, and wherein the weight ratio of a to b is from about 0.4 to about 4, and wherein the total concentration of a and b combined is about 10 to about 20% of the total weight of the composition.

In some embodiments, provided is a coacervate cleansing system, comprising:
(a) at least one amphoteric surfactant selected from the group consisting of cocamidopropyl hydroxysultaine and coco betaine from about 3% to about 8% by weight, relative to the total weight of the composition;
(b) at least one glycolipid selected from the group consisting of rhamnolipid and sophorolipid from about 2% to about 7% by weight, relative to the total weight of the composition;
(c) at least one cationic polymer selected from the group consisting of chitosan and polylysine from about 0.1% to about 0.2%;
(d) at least one mineral particle selected from the group consisting of titanium dioxide and zinc oxide from about 1% to about 10%; and
(e) water;
   wherein the cleansing system is essentially free of phenoxyethanol, and wherein the pH of the composition is between 4 and 8, and wherein the weight ratio of a to b is about 0.4 to about 4.

In some embodiments, provided is a method for depositing at least one mineral particle to the skin during cleansing comprising (i) applying to the skin of a subject a coacervate cleansing system, comprising:
(a) at least one amphoteric surfactant;
(b) at least one glycolipid;
(c) at least one cationic polymer from about 0.05% to about 2%;
(d) at least one mineral particle; and
(e) at least one cosmetically acceptable solvent;
wherein the cleansing system is essentially free of phenoxyethanol, and wherein the pH of the composition is between 4 and 8, and wherein the weight ratio of a to b is about 0.4 to about 4, and wherein the total concentration of a and b combined is about 10% to about 20% of the total weight of the composition, and (ii) removing the cleansing system from the skin.

The coacervate system described in this invention is a unique phenomenon that demonstrates enhanced stability which is beneficial when cleansing the skin. The coacervate phenomenon is demonstrated through the phase transition of the system by initially appearing clear, but then appearing cloudy upon dilution with water. This coacervate cleansing systems provides a unique cleansing experience marked by enhanced foaming properties and good stability.

The coacervate cleansing system described has unique physical characteristics, and transitions into a foaming state when diluted with water which could enhance deposition of skincare actives onto the skin. It has surprisingly been found that compositions with the above disclosed ratio of glycolipids to amphoteric surfactants demonstrate enhanced deposition of TiO2 particles onto the skin.

### BRIEF DESCRIPTION OF THE FIGURES

FIG. 1. Coacervate cleansing system compositions A-D before dilution.

FIG. 2. Coacervate cleansing system compositions A-D after dilution with water.

FIG. 3. Coacervate cleansing system containing TiO2 composition 19 before being rinsed off.

FIG. 4. Coacervate cleaning system containing TiO2 composition 19 after being rinsed off.

FIG. 5. Non-inventive composition 20 after being rinsed off.

FIG. 6. Non-inventive composition 21 after being rinsed off.

It is to be understood that the foregoing and following descriptions are exemplary and explanatory only, and are not intended to be restrictive of any subject matter claimed.

### DETAILED DESCRIPTION

The disclosure relates to compositions for depositing mineral particles to the skin after cleansing skin and methods of using the compositions.

### I. Compositions

### Amphoteric Surfactants

In the various embodiments, the cosmetic cleansing composition comprises at least one amphoteric surfactant (or zwitterionic surfactant). In some embodiments, the cosmetic cleansing composition may include or excludes any one or more of nonionic, cationic, or anionic surfactants. In some embodiments, the cosmetic cleansing composition includes a combination of amphoteric surfactants.

In some embodiments, the at least one amphoteric surfactant may include alkyl betaine, alkylamidopropyl betaine, alkyl hydroxysultaine, alkylamidopropyl hydroxysultaine, lauryl hydroxysultaine, alkylamidopropylamine N-oxide, alkyldimethylamine N-oxide, cocamidopropyl hydroxysultaine, cocamidopropyl betaine, coco betaine, sodium lauroamphoacetate, disodium cocoamphodiacetate, or a combination thereof. In preferred embodiments, the amphoteric surfactant is selected from cocamidopropyl hydroxysultaine and coco betaine.

In some embodiments the at least one amphoteric surfactant may be selected from, for example, betaines, alkyl sultaines, alkyl amphoacetates and alkyl amphodiacetates, alkyl amphoproprionates, amphocarboxylates, alkyl betaines, amidoalkyl betaines, amphophosphates, phosphobetaines, pyrophosphobetaines, carboxyalkyl polyamines, amidoalkyl sultaines, salts thereof, or mixtures thereof.

Betaines which can be used in the current compositions include those having the formulas below:

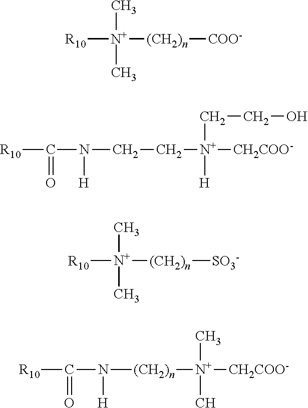

wherein
   R¹⁰ is an alkyl group having 8-18 carbon atoms; and
   n is an integer from 1 to 3.

Particularly useful betaines include, for example, cocoamidopropyl hydroxysultaine, coco betaine, cocoamidopropyl betaine, lauryl betaine, laurylhydroxy sulfobetaine, lauryldimethyl betaine, behenyl betaine, capryl/capramidopropyl betaine, lauryl hydroxysultaine, stearyl betaine, and mixtures thereof.

Hydroxyl sultaines useful in the compositions of the invention include the following

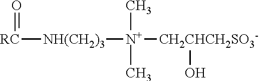

wherein
   R is an alkyl group having 8-18 carbon atoms.

More specific examples include, but are not limited to cocamidopropyl hydroxysultaine, lauryl hydroxysultaine, or mixtures thereof.

Useful alkylamphoacetates include those having the formula

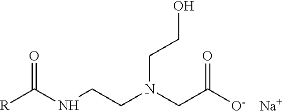

wherein
R is an alkyl group having 8-18 carbon atoms.
useful alkyl amphodiacetates include those having the formula

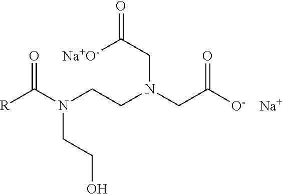

wherein
R is an alkyl group having 8-18 carbon atoms.

Exemplary and non-limiting examples of useful alkyl amphopropionates include cocoamphopropionate, caprylamphopropionate, cornamphopropionate, caproampho-propionate, oleoamphopropionate, isostearoamphopropionate, stearoamphopropionate, lauroamphopropionate, salts thereof, or mixtures thereof.

The at least one amphoteric surfactant of the present disclosure may be optionally quaternized secondary or tertiary aliphatic amine derivatives, in which the aliphatic group is a linear or branched chain comprising from 8 to 22 carbon atoms, said amine derivatives containing at least one anionic group, for instance a carboxylate, sulfonate, sulfate, phosphate or phosphonate group.

In preferred embodiments, the at least one amphoteric surfactant is chosen from cocamidopropyl hydroxysultaine, coco betaine, or mixtures thereof.

The total concentration of the at least one amphoteric surfactant and the at least one glycolipid is about 10% to about 20% of the total weight of the composition. The weight ratio of the at least one amphoteric surfactant to the at least one glycolipid is from about 0.4 to about 4. The at least one amphoteric surfactant may be present in the cosmetic cleansing composition, for example, in a range for example from about 3% to about 8%, based on the weight of the cosmetic cleansing composition.

For example, the at least one amphoteric surfactant can be present in the cosmetic cleansing composition according to the disclosure from about 3% to about 8%, or from about 3% to about 7%, or from about 3% to about 6%, or from about 3% to about 5%, or from about 3% to about 4%, or from about 3% to about 8%, or from about 4% to about 8%, or from about 5% to about 8%, or from about 6% to about 8%, or from about 7% to about 8%, or any suitable combination, sub-combination, range, or sub-range thereof by weight, based on the weight of the cosmetic cleansing composition. One of ordinary skill in the art, however, will appreciate that other ranges are within the scope of the invention.

### Glycolipids

The composition according to the invention comprises one or more glycolipids.

The term “glycolipid” is understood as meaning a compound formed from a lipid to which are attached one or more sugar compounds.

The one or more glycolipids may be selected from rhamnolipids, sophorolipids, glucolipids, trehalolipids, cellobiose lipids, mannosylerythritol lipid, and mixtures thereof.

### Glucolipids

The one or more glycolipids may be glucolipids, which contain a glucose moiety and can be represented by the general formula (I):

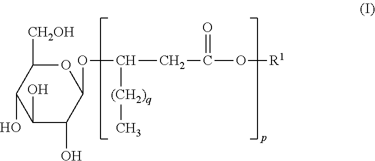

in which:
   R1 represents a hydrogen atom or a cation,
   p denotes an integer ranging from 1 to 4, and
   q denotes an integer ranging from 4 to 10, preferably equal to 6.

The glucolipids can be produced by the bacterium Alcaligenes sp. MM1.

The appropriate fermentation methods are reviewed by M. Schmidt in his doctoral thesis (1990), Technical University of Braunschweig, and by Schulz et al. (1991) Z. Naturforsch., 46C, 197-203. The glucolipids are recovered from the fermentation broth by solvent extraction using diethyl ether or a dichloromethane:methanol or chloroform:methanol mixture.

### Sophorolipids

The one or more glycolipids may be sophorolipids, which contain a sophorose moiety and can be represented by the general formula (II):

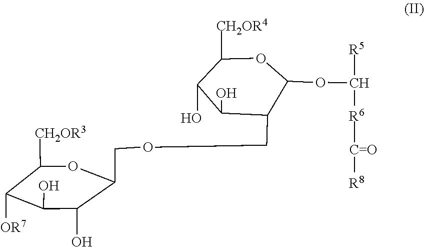

in which:
   R3 and R4 individually represent a hydrogen atom or an acetyl group,
   R5 represents a saturated or unsaturated, hydroxylated or non-hydroxylated hydrocarbon group having from 1 to 9 carbon atoms, preferably methyl,
   R6 represents a saturated or unsaturated, hydroxylated or non-hydroxylated hydrocarbon group having from 1 to 19 carbon atoms, with the proviso that the total number of carbon atoms in the groups R5 and R6 does not exceed 20 and is preferably from 14 to 18.

Sophorolipids may be incorporated into the composition according to the invention either in the form of the open-chain free acid, where R7 represents a hydrogen atom and R8 represents a hydroxy group OH, or in its lactone form, where a lactone ring is formed between R7 and R8, as indicated by formula (III):

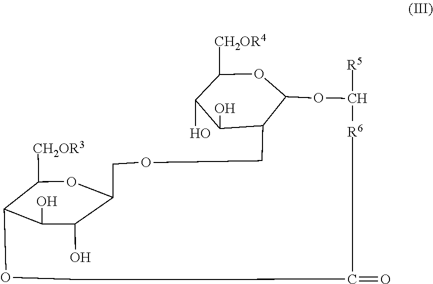

in which:
   R3, R4, R5 and R6 are as defined above,
with the proviso that at least one of R3 and R4 represents an acetyl group.

The sophorolipids can be produced by yeast cells, for example Torulopsis apicola and Torulopsis bombicola cells. The fermentation process generally uses sugars and alkanes as substrates.

Appropriate fermentation methods are reviewed in A. P. Tulloch, J. F. T. Spencer and P. A. J. Gorin, Can. J. Chem. (1962), 40, 1326, and U. Gobbert, S. Lang and F. Wagner, Biotechnology Letters (1984), 6 (4), 225. The resulting product is a mixture of various open-chain sophorolipids and of sophorolipid lactones that may be used in the form of mixtures, or the required form may be isolated.

It is possible to use as sophorolipids for example that sold under the Sopholiance S name by Givaudan and that sold under the BioToLife name by BASF.

### Trehalolipids

The one or more glycolipids may be trehalolipids, which contain a trehalose fragment and can be represented by the general formula (IV):

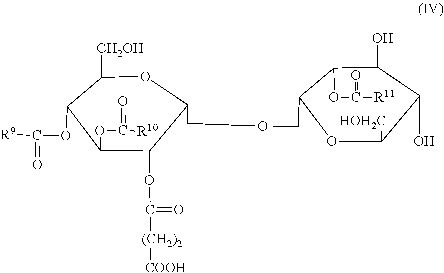

in which:
   R9, R10 and R11 individually represent a saturated or unsaturated, hydroxylated or non-hydroxylated hydrocarbon radical having from 5 to 13 carbon atoms.

The trehalolipids can be produced by bacterial fermentation using the marine bacterium Arthrobacter sp. Ek 1 or the freshwater bacterium Rhodococcus erythropolis. Appropriate fermentation methods are provided by Ishigami et al. (1987), J. Jpn. Oil Chem. Soc., 36, 847-851, Schultz et al. (1991), Z. Naturforsch., 46C, 197-203, and Passeri et al. (1991), Z. Naturforsch., 46C, 204-209.

### Cellobiose Lipids

The one or more glycolipids may be cellobiose lipids, which contain a cellobiose fragment and can be represented by the general formula (V):

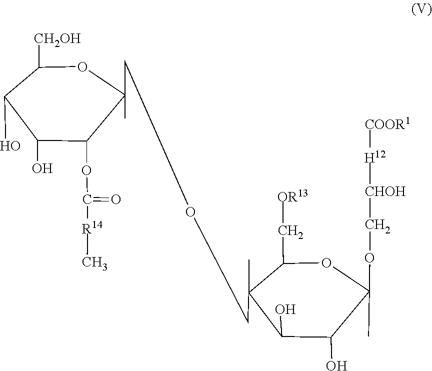

in which:
   R1 represents a hydrogen atom or a cation,
   R12 represents a saturated or unsaturated, hydroxylated or non-hydroxylated hydrocarbon radical having from 9 to 15 carbon atoms, preferably 13 carbon atoms,
   R13 represents a hydrogen atom or a acetyl group;
   R14 represents a saturated or unsaturated, hydroxylated or non-hydroxylated hydrocarbon radical having from 4 to 16 carbon atoms.

The cellobiose lipids can be produced by cells of fungi of the genus Ustilago. Appropriate fermentation processes are provided by Frautz, Lang and Wagner (1986), Biotech. Letts., 8, 757-762.

### Rhamnolipids

The one or more glycolipids may be rhamnolipids.

The composition according to the invention preferably comprises one or more rhamnolipids.

Rhamnolipids are glycolipids produced by various bacterial species. They consist of one rhamnose fragment (mono-rhamnolipid) or of two rhamnose fragments (di-rhamnolipid) linked by a glycosidic bond to one, two or three chains of β-hydroxylated fatty acids linked to one another by an ester bond.

More specifically, these mono-rhamnolipids and di-rhamnolipids correspond to the following formula (VI):

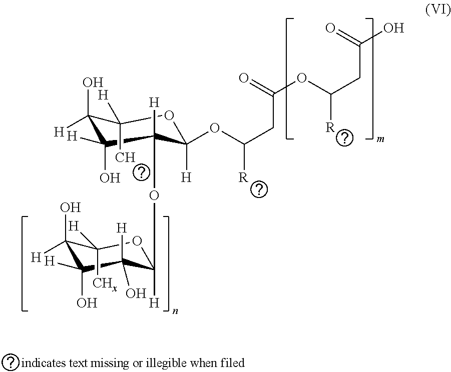

in which:
   m denotes an integer equal to 2, 1 or 0,
   n denotes an integer equal to 1 or 0, and
   R1 and R2, each independently represent identical or different hydrocarbon radicals having from 2 to 24 carbon atoms, preferably from 5 to 13 carbon atoms, that are branched or unbranched, substituted or unsubstituted, in particular hydroxy-substituted, and saturated or unsaturated, preferably a singly, doubly or triply unsaturated alkyl radical.

Thus, when n is equal to 0, the formula (VI) protects mono-rhamnolipids and, when n is equal to 1, it protects di-rhamnolipids.

The composition according to the invention preferably comprises at least one di-rhamnolipid.

The composition according to the invention preferably comprises at least one di-rhamnolipid of formula (VI) in which:
m denotes an integer equal to 2, 1 or 0;
n denotes an integer equal to 1; and
R1 and R2, each independently represent identical or different hydrocarbon radicals having from 2 to 24 carbon atoms, preferably from 5 to 13 carbon atoms, that are branched or unbranched, substituted or unsubstituted, in particular hydroxy-substituted, and saturated or unsaturated, preferably a singly, doubly or triply unsaturated alkyl radical, and also the salts thereof, solvates thereof and optical isomers thereof.

The glycosidic bond between the two rhamnose fragments may be in the alpha or beta configuration and is preferably in the alpha configuration.

In the context of the invention,
the salts of the di-rhamnolipids of formula (VI) are more particularly the carboxylate salts thereof with an organic or inorganic cation and especially with a cation selected from sodium, potassium, calcium and ammonium.
the solvated forms of the di-rhamnolipids of formula (VI) are more particularly those solvated with one or more molecules of water or of organic solvents, for example a hydrate or a solvate of a linear or branched alcohol, such as ethanol or isopropanol, the optically active carbon atoms of the fatty acids preferably being in the form of the R enantiomers, and
the term “alkyl” radical denotes a saturated, linear or branched aliphatic group; for example, a C1-C20 alkyl group having a linear or branched hydrocarbon chain of 1 to 20 carbon atoms, more particularly a methyl, ethyl, propyl, isopropyl, butyl, isobutyl, tert-butyl, pentyl, hexyl, heptyl, octyl, nonyl, decyl, undecyl, dodecyl, tridecyl, tetradecyl, pentadecyl, hexadecyl, heptadecyl, octadecyl, nonadecyl or eicosyl.

The composition according to the invention preferably comprises at least one di-rhamnolipid of formula (VI) in which:
m denotes an integer equal to 2, 1 or 0;
n denotes an integer equal to 1; and
R1 and R2, which are identical or different, are selected from pentenyl, hexenyl, heptenyl, octenyl, nonenyl, decenyl, undecenyl, dodecenyl and tridecenyl radicals and radicals of formula —(CH2)oCH3, with o denoting an integer ranging from 1 to 23, in particular from 3 to 15 and more particularly from 4 to 12.

According to one embodiment of the invention, the composition according to the invention comprises at least one di-rhamnolipids of general formula (VI) in which m is equal to 1

According to one embodiment of the invention, the composition according to the invention comprises a mixture of at least two, preferably at least three, di-rhamnolipids of general formula (VI) in which m is preferably equal to 1.

According to another embodiment of the invention, the composition according to the invention comprises a mixture comprising at least one mono-rhamnolipid.

More preferably, the composition according to the invention comprises at least one dirhamnolipid of the following formula (VII):

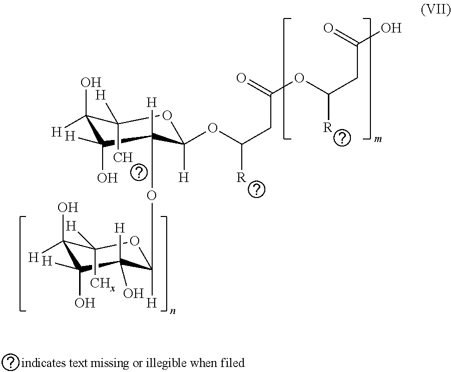

in which:
   m denotes an integer equal to 2, 1 or 0; preferably, m is equal to 1,
   n denotes an integer equal to 1,
   R1 is a —(CH2)p—CH3 radical, with p being an integer varying from 1 to 23, preferably from 4 to 12,
   R2 is a —(CH2)q—CH3 radical, with q being an integer varying from 1 to 23, preferably from 4 to 12,
and also the salts thereof, solvates thereof and optical isomers thereof.

By way of illustration and without limiting the di-rhamnolipids of formula (VII) that may be suitable for the invention, mention may be made in particular of the compounds of formula di-RL-CXCY, such as are defined in Table 1 below.

The formula di-RL-CXCY is an alternative way of writing in order to represent a di-rhamnolipid (di-RL) functionalized by two radicals R1 and R2 respectively represented by the symbols CX and CY, the integers X and Y being respectively equal to p+4 and q+4.

TABLE 1
di-rhamnolipids of Formula (VII)

| Composes | Di-RL-CXCY | p | q |
|---|---|---|---|
| 1 | diRL-C8C8 | 4 | 4 |
| 2 | diRL-C8C10 | 4 | 6 |
| 3 | diRL-C10C8 | 6 | 4 |
| 4 | diRL-C10C10 | 6 | 6 |
| 5 | diRL-C10C12 | 6 | 8 |
| 6 | diRL-C12C10 | 8 | 6 |
| 7 | diRL-C12C12 | 8 | 8 |
| 8 | diRL-C12C14 | 8 | 10 |
| 9 | diRL-C14C12 | 10 | 8 |
| 10 | diRL-C14C14 | 10 | 10 |
| 11 | diRL-C14C16 | 10 | 12 |
| 12 | diRL-C16C14 | 12 | 10 |
| 13 | diRL-C16C16 | 12 | 12 |

According to a preferred embodiment, the composition according to the invention comprises at least one di-rhamnolipid of formula (VII) in which p and q are identical and equal to 6 and m is equal to 1, also referred to as di-RL-C10C10, or one of the salts, solvates and optical isomers thereof.

Preferably, the di-rhamnolipid of formula (VII) in which p and q are identical and equal to 6 and m is equal to 1 is present in the composition according to the invention in a proportion of at least 50% by weight and preferably of from 51% to 85% by weight, relative to the total weight of rhamnolipids.

According to another embodiment, the composition according to the invention comprises at least one di-rhamnolipid of formula (VII) in which m is equal to 1, p is equal to 6 and q is equal to 8.

According to another embodiment, the composition according to the invention comprises at least one di-rhamnolipid of formula (VI) in which n and m are equal to 1, R1 represents a —(CH2)oCH3 radical, with o being an integer varying from 4 to 12, and R2 is selected from the pentenyl, hexenyl, heptenyl, octenyl, nonenyl, decenyl, undecenyl, dodecenyl and tridecenyl radicals; preferably, R1 represents a —(CH2)6CH3 radical and R2 a nonenyl radical.

According to another preferred embodiment, the composition according to the invention comprises a mixture of at least two, in particular at least three, di-rhamnolipids of formula (VI) or of formula (VII) selected from:
a di-rhamnolipid of formula (VII) in which p and q are identical and equal to 6 and m is equal to 1;
a di-rhamnolipid of formula (VII) in which m is equal to 1, p is equal to 6 and q is equal to 8; and
at least one di-rhamnolipid of formula (VI) in which n and m are equal to 1, R1 represents a —(CH2)oCH3 radical, with o being an integer varying from 4 to 12, and R2 is selected from the pentenyl, hexenyl, heptenyl, octenyl, nonenyl, decenyl, undecenyl, dodecenyl and tridecenyl radicals; preferably, R1 represents a —(CH2)6CH3 radical and R2 a nonenyl radical.

Preferably, the composition according to the invention comprises a mixture of at least two, in particular at least three, di-rhamnolipids of formula (VI) or of formula (VII) selected from:
at least 50% by weight and preferably of from 51% to 85% by weight of a di-rhamnolipid of formula (VII) in which p and q are identical and equal to 6 and m is equal to 1, relative to the total weight of rhamnolipids.
from 0.5% to 25% by weight, preferably from 5% to 15% by weight, of a dirhamnolipid of formula (VII) in which p is equal to 6, q is equal to 8 and m is equal to 1, relative to the total weight of rhamnolipids, and
from 0.5% to 15% by weight, preferably from 3% to 12% by weight, preferably from 5% to 10% by weight, of a dirhamnolipid of formula (VI) in which n and m are equal to 1, R1 represents a —(CH2)6CH3 radical and R2 represents a nonenyl radical, relative to the total weight of rhamnolipids.

As specified above, rhamnolipids are customarily prepared by processes known to those skilled in the art starting from bacterial producers, such as Pseudomonas.

Appropriate fermentation methods are reviewed by D. Haferburg, R. Hommel, R. Claus and H. P. Kleber in Adv. Biochem. Ing./Biotechnol. (1986), 33, 53-90, and by F. Wagner, H. Bock and A. Kretschmar in Fermentation (ed. R. M. Lafferty) (1981), 181-192, Springer Verlag, Vienna.

Use may be made, as rhamnolipid, of the one sold under the name Rheance One by Evonik (INCI name: glycolipids).

In preferred embodiments, the at least one glycolipid is chosen from rhamnolipids, sophorolipids, or mixtures thereof.

The total concentration of the at least one amphoteric surfactant and the at least one glycolipid is about 10% to about 20% of the total weight of the composition. The weight ratio of the at least one amphoteric surfactant to the at least one glycolipid is from about 0.4 to about 4. The at least one glycolipid may be present in the cosmetic cleansing composition, for example, in a range from about 3% to about 7%, based on the weight of the cosmetic cleansing composition.

For example, the at least one glycolipid may be present in the cosmetic cleansing composition according to the disclosure from about from about 3% to about 7%, or from about 3% to about 6%, or from about 3% to about 5%, or from about 3% to about 4%, or from about 3% to about 7%, or from about 4% to about 7%, or from about 5% to about 7%, or from about 6% to about 7%, or any suitable combination, sub-combination, range, or sub-range thereof by weight, based on the weight of the cosmetic cleansing composition. One of ordinary skill in the art, however, will appreciate that other ranges are within the scope of the invention.

### Cationic Polymer

In the various embodiments, the cosmetic cleansing composition comprises at least one cationic polymer selected from nature-based polymers that are polysaccharides, and other natural (i.e., plant, animal, or bacterial based), synthetic, or modified cationic nature-based or synthetic polymers.

In some embodiments, the cationic polymer is a nature-based polymer. In general, a cationic nature-based polymer may be selected from cationic forms and cationic derivatives of polysaccharides isolated from algae, polysaccharides produced by microorganisms, and polysaccharides from higher plants, such as homogeneous polysaccharides.

In some embodiments, cationic nature-based polymer selected from polysaccharides may be chosen from polysaccharides that include chitosan, chitin, starches, alginates, celluloses, galactomannans such as guar gums, particularly cationic derivatives thereof, and combinations thereof. In some embodiments, the at least one cationic nature-based polymer selected from polysaccharides may be chosen from chitosan, chitosan derivatives, chitin, starch, starch derivatives, cellulose (for example, but not limited to, ethylcellulose, nitrocellulose, hemicellulose, and hemicellulose derivatives), alginates, including but not limited to, sodium alginate, and combinations thereof.

In some embodiments, the cationic polymer is a nature-based cationic polymer selected from: chitosan, chitosan oligosaccharide, polymeric chitosan having MW in a range from 1 kDa to about 1000 kDa, derivatives of chitosan, derivatives of chitosan having enhanced solubility, cyclodextrin, cationic gelatin, cationic dextran, cationic cellulose, polylysine, polyornithine, histone, collagen, chitosan-cysteine, chitosan-thiobutylamidine, chitosan-thioglycolic acid, or combinations thereof. In preferred embodiments, the cationic polymer is selected from chitosan, polylysine, or combinations thereof.

Examples of cationic polymers include polysaccharide-based delivery molecules (e.g., chitosan, cyclodextrin, cationic gelatin, cationic dextran, cationic cellulose), cationic peptides and their derivatives (e.g., polylysine, polyornithine), peptide/protein polymers ((e.g., histone, collagen), linear or branched synthetic polymers (e.g., polybrene, polyethyleneimine), natural polymers (e.g., histone, collagen), synthetic dendrimers, cationic thiolated biopolymers (nature-based thiomers or nature-based dendrimers, e.g., chitosan-cysteine, chitosan-thiobutylamidine as well as chitosan-thioglycolic acid).

Examples of nature-based cationic polymers include polysaccharide-based delivery molecules (e.g., chitosan, cyclodextrin, cationic gelatin, cationic dextran, cationic cellulose); cationic peptides and their derivatives (e.g., polylysine, polyornithine), peptide/protein polymers (e.g., histone, collagen); cationic thiolated biopolymers (nature-based thiomers or nature-based dendrimers, e.g., chitosan-cysteine, chitosan-thiobutylamidine, chitosan-thioglycolic acid); or combinations thereof.

In some embodiments, the cosmetic cleansing composition comprises the at least one nature-based cationic chitosan selected from chitosan oligosaccharide, chitosan (or polymeric chitosan having molecular weight (MW) in a range from about 1 kDa to about 1000 kDa), derivatives of chitosan, including derivatives having enhanced solubility, or combinations thereof.

In various embodiments, chitosan has a molecular weight (MW) in a range from about 1 kDa to about 1000 kDa. In some particular embodiments, chitosan has a MW that is “low” and is in the range from about 1 kDa to about 20 kDa, or from about 10 kDa to about 20 kDa, or from about 12 kDa to about 18 kDa. In some embodiments, the chitosan has a Chitosan MW=˜27 kDa

According to the present invention, the cationic polymer may be selected from polylysines. A single type of polylysines may be used, or two or more different types of polylysines may be used in combination.

Polylysines correspond to the condensation of several amino acids of lysine. Polylysine can be a natural homopolymer of L-lysine that can be produced by bacterial fermentation. Polylysines are typically used as a natural preservative in food products. Polylysine is a polyelectrolyte which is soluble in polar solvents such as water.

Polylysine can be, for example, epsilon-polylysine (or referred as “ε-polylysine”), which is a condensation of amino groups at the ε-position and carboxyl groups of lysines, or alpha-polylysine (or referred as “α-polylysine”), which is a condensation of amino groups at the a-position and carboxyl groups of lysines. Polylysine is commercially available in various forms, such as poly D-lysine and poly L-lysine. The polylysine is generally a condensate of L-lysines, i.e., poly L-lysine.

As an example of polylysine, mention may be made of:
Epsilon-poly-L-lysine of JNC CORPORATION which is a 25% solution of Epsilon-poly-L-lysine having a molecular weight of around 4,700 in aqueous solution.

The polylysine may be in the form of organic or inorganic salts. The addition salts with an acid are, for example, the hydrochloric or hydrobromic acid, sulfuric acid, citric acid, succinic acid, tartaric acid, lactic acid, para-toluenesulphonic acid, phosphoric acid, or acetic acid salts; or fatty acid salts, such as linoleic acid, oleic acid, palmitic acid, stearic acid, behenic acid, and 18-methylicosanoic acid. The addition salts with a base are, for example, a sodium salt, a calcium salt, or a hydroxyalkylamine salt, for example, N-methylglucamine, aminopropane diol or triethanolamine.

In some preferred embodiments of the present invention, the polylysine of the present invention is present in a form of a single molecule in the composition, or is not covalently bound to other compounds. In one embodiment of the present invention, the polylysine is not covalently bound to dye compounds. In one embodiment of the present invention, the polylysine is not covalently bound to polyorganosiloxane compounds. The term “polyorganosiloxane” is well-known in the art to mean compounds having Si—O main chain and organic functional groups attached to the main chain.

In another embodiment of the present invention, the polylysine is in the free form. The term “free form” here indicates that the polylysine is not covalently bound to any other compounds.

The at least one nature-based cationic polymer is present in the cosmetic cleansing composition according to the disclosure from about 0.05% to about 2%, or from about 0.05% to about 1%, or from about 0.05% to about 1%, or from about 0.1% to about 0.5%, or from about 0.1% to about 0.2%, or any suitable combination, sub-combination, range, or sub-range thereof by weight, based on the weight of the cosmetic cleansing composition. One of ordinary skill in the art, however, will appreciate that other ranges are within the scope of the invention.

Thus, one or a combination of polymers may be present, by weight, based on the total weight of the cosmetic cleansing composition, from about 0.05, 0.06, 0.07, 0.08, 0.90, 0.1, 0.2, 0.3, 0.4, 0.5, 0.6, 0.7, 0.8, 0.9, 1.0, to about 2 weight percent, including increments and ranges therein and there between.

In other embodiments, cationic nature-based polymer selected from polysaccharides may be chosen from one or more of methylcelluloses, hydroxyalkylcelluloses, ethylhydroxyethylcelluloses and carboxymethylcelluloses, mannans, xylans, lignins, arabans, galacturonans, alginate-based compounds, chitin, glucuronoxylans, arabinoxylans, xyloglucans, glucomannans, fructosans such as inulin, pectic acids and pectins, arabinogalactans, agars, glycosaminoglycans, gum arabics, tragacanth gums, ghatti gums, karaya gums, locust bean gums, biopolysaccharide gums of microbial origin such as scleroglucan or xanthan gums, mucopolysaccharides, chondroitin sulfates, and mixtures thereof.

In some embodiments, the cationic polymer is not a nature-based polymer and may be selected from synthetic polymers. In some examples, the synthetic polymers are non-silicone based, and in some embodiments may be selected from the group consisting of polyquaterniums, that is polymers having quaternary ammonium centers in the polymer, for example, including polyquaternium-47 (1-Propanaminium, N,N,N-trimethyl-3-[(2-methyl-1-oxo-2-propenyl)amino]-, chloride, polymer with methyl 2-propenoate and 2-propenoic acid). Other non-limiting examples of such polyquaternium compounds may be selected from diallyidimethylammonium chloride/acrylic acid copolymers sold under the names MERQUAT 280 POLYMER or MERQUAT 280NP POLYMER or MERQUAT 281 POLYMER or MERQUAT 295 POLYMER, by the company Nalco (Lubrizol) (INCI name: Polyquaternium-22); the copolymer of methacrylamidopropyltrimonium chloride, of acrylic acid and or methyl acrylate, sold under the name MERQUAT 2001 POLYMER OR MERQUAT 2001N POLYMER by the company Nalco (Lubrizol) (INCI name: Polyquaternium-47); the acrylamide/dimethyldiallylammonium chloride/acrylic acid terpolymer sold under the name MERQUAT 3330DRY POLYMER or MERQUAT 3330PR POLYMER or MERQUAT 3331PR POLYMER or MERQUAT 3940 POLYMER or MERQUAT PLUS 3330 POLYMER OR MERQUAT PLUS 3331 POLYMER by the company Nalco (Lubrizol) (INCI name:: Polyquaternium-39); an ampholytic terpolymer consisting of methacrylamidopropyl trimethyl ammonium chloride (MAPTAC), acrylamide and acrylic acid, sold under the name MERQUAT 2003PR POLYMER by the company Nalco (Lubrizol) (INCI name:: Polyquaternium-53); Polyquaternium-30, Polyquaternium-35, Polyquaternium-45, Polyquaternium-50, Polyquaternium-54; Polyquaternium-57; Polyquaternium-63; Polyquaternium-74; Polyquaternium-76; Polyquaternium-86; Polyquaternium-89; Polyquaternium-95; Polyquaternium-98, Polyquaternium-104; Polyquaternium-111; Polyquaternium-112, and mixtures thereof. In some embodiments, the cationic polymer is a synthetic polymer selected from cationic acrylic polymers, for example, an amorphous functional acrylic polymer grafted onto a polyethylene backbone such as SYNTRAN™ 5330, which is a quaternary modified olefin grafted technology. More generally, such synthetic polymers selected from, but are not limited to, cationic polymers comprising polyacrylates such as those identified in the International Cosmetic Ingredient Dictionary and Handbook (9 th ed. 2002) such as, for example, polyacrylate-1, polyacrylate-2, polyacrylate-3, polyacrylate-4, polyacrylate-16, polyacrylate-17, polyacrylate-18, polyacrylate-19, polyacrylate-21, and mixtures thereof. Such (co)polymers, or similar (co)polymers, can be combined individually or with other (co)polymers in such a way to form suitable bimodal agents having both cationic and anionic functionalities.

In the various embodiments, the amount of cationic polymer is not a nature-based polymer and is selected from synthetic cationic polymers that may be present in the cosmetic cleansing composition can range from about 0.05% to about 2%, or from about 0.05% to about 1%, or from about 0.05% to about 1%, or from about 0.1% to about 0.5%, or from about 0.1% to about 0.2%, or any suitable combination, sub-combination, range, or sub-range thereof by weight, based on the weight of the cosmetic cleansing composition.

Thus, any one of the at least synthetic cationic polymer, when present, is present, by weight, based on the total weight of the cosmetic cleansing composition, from about 0.05, 0.06, 0.07, 0.08, 0.09, 0.1, 0.2, 0.3, 0.4, 0.5, 0.6, 0.7, 0.8, 0.9, 1, to about 2 weight percent, including increments and ranges therein and there between.

In the various embodiments, the amount of each of the at least one cationic polymer which may be either nature-based or synthetic cationic polymer, alone or in combination with another cationic polymer, present in the cosmetic cleansing compositions can range from about 0.05% to about 2%, or from about 0.05% to about 1%, or from about 0.05% to about 1%, or from about 0.1% to about 0.5%, or from about 0.1% to about 0.2% or any suitable combination, sub-combination, range, or sub-range thereof by weight, based on the weight of the cosmetic cleansing composition. One of ordinary skill in the art, however, will appreciate that other ranges are within the scope of the invention.

In preferred embodiments, according to the invention the cationic polymer is chosen from nature based cationic polymers.

Compositions according to the disclosure contain at least one cosmetically acceptable solvent. In various embodiments, the cosmetically acceptable solvent may be chosen from water.

Cosmetic cleansing compositions according to the disclosure are essentially free of phenoxyethanol.

In various embodiments, the compositions have a pH from about 4 to about 8. For example, the pH of the compositions may range from about 4.5 to about 7.5, including all ranges and subranges therebetween.

### Mineral Particles

In the various embodiments, the coacervate cleansing system comprises at least one mineral particle selected from mineral pigments or mineral UV filters.

### Mineral Pigments

The term “mineral pigment” is intended to mean any pigment that satisfies the definition in Ullmann's encyclopaedia in the chapter on inorganic pigments. Among the mineral pigments that are useful in the present invention, mention may be made of zirconium oxide, cerium oxide, zinc oxide, chromium oxide, titanium dioxide, or metal powders, for instance aluminium powder and copper powder. The following mineral pigments may also be used: Ta₂O₅, Ti₃O₅, Ti₂O₃, TiO, ZrO₂ as a mixture with TiO₂, ZrO₂, Nb₂O₅, CeO₂, ZnS.

In the context of the present invention, the mineral pigments are more particularly iron oxide and/or titanium dioxide.

In some embodiments, the one or more pigments comprises pigment-grade titanium dioxide. “Pigment-grade” titanium dioxide generally has a larger particle size than UV grade titanium dioxide-typically having mean particle sizes of 400 nm or larger, including 500 nm or larger, and/or 600 nm or larger.

In some particular embodiments, the amount of at least one mineral pigment is present from about 1% to about 10 wt. %, based on the total weight of the composition. The total amount of mineral pigments in the compositions can vary but is typically from about 1,2,3,4,5, 6, 7, 8, 9, or 10 wt. % based on the total weight of the composition.

### Mineral UV Filters

In various embodiments, the composition comprises one or a combination of mineral UV filters. The mineral UV filter used for the present disclosure may be active in the UV-A and/or UV-B region. The mineral UV filter may be hydrophilic and/or lipophilic.

In preferred embodiments, the mineral UV filter is in the form of a fine particle such that the mean (primary) particle diameter thereof ranges from 1 nm to 150 nm, and in some embodiments 5 nm to 40 nm, and in some embodiments 10 nm to 30 nm. The mean (primary) particle size or mean (primary) particle diameter herein is an arithmetic mean diameter. In some embodiments, a particle may have a diameter that is has a mean particle size that is about or less than about 1 micron, or that is about or less than about 200 nm, or that is about or less than about 100 nm (sometimes referred to nanoscale). It will be understood by one of ordinary skill in the art that a particle that is referred to as nano scale for purposes of cosmetic applications according to this disclosure will have a mean particle size that is less than about 100 nm unless specifically identified otherwise, for example as in the case of nano zinc oxide which is sometimes referred to as nanoscale though the UV agent has a mean primary particle size that is less than 200 nm.

In some particular embodiments, the amount of at least one mineral UV filter is present from about 1% to about 10 wt. %, based on the total weight of the composition. The total amount of mineral UV filtering agents in the compositions can vary but is typically from about 1,2,3,4,5, 6, 7, 8, 9, or 10 wt. % based on the total weight of the composition.

The mineral UV filter can be selected from the group consisting of metal oxides, which may or may not be coated. And in some embodiments, the mineral UV filters are selected from pigments formed of metal oxides, such as, for example, pigments formed of titanium dioxide (amorphous or crystalline in the rutile and/or anatase form), iron oxide, zinc oxide, zirconium oxide, or cerium oxide, which are all UV photoprotective agents that are well known per se.

The mineral UV filter may or may not be coated, and said coated mineral UV filter may have one or more coating. The coating may comprise one or more compound selected from the group consisting of alumina, silica, aluminum hydroxide, silicones, silanes, fatty acids or salts thereof (such as sodium, potassium, zinc, iron, or aluminum salts), fatty alcohols, lecithin, amino acids, polysaccharides, proteins, alkanolamines, waxes, such as beeswax, (meth)acrylic polymers, organic UV filters, and (per)fluoro compounds. It is in some embodiments desirable for the coating to include one or a combination of organic UV filter.

Of course, the mineral UV filter made of metal oxides may, before their treatment with silicones, have been treated with other surfacing agents, in particular, with cerium oxide, alumina, silica, aluminum compounds, silicon compounds, or their mixtures. The coated mineral UV filter may have been prepared by subjecting the mineral UV filter to one or more surface treatments of a chemical, electronic, mechano-chemical, and/or mechanical nature with any of the compounds as described above, as well as polyethylenes, metal alkoxides (titanium or aluminum alkoxides), metal oxides, sodium hexametaphosphate, and those shown, for example, in Cosmetics & Toiletries, February 1990, Vol. 105, pp. 53-64.

The coated mineral UV filter may be titanium oxides coated: with silica, such as the product “Sun veil” from Ikeda, and “Sunsil TIN 50” from Sunjin Chemical; with silica and with iron oxide, such as the product “Sunveil F” from Ikeda; with silica and with alumina, such as the products “Microtitanium Dioxide MT 500 SA” from Tayca, “Tioveil” from Tioxide, and “Mirasun TiW 60” from Rhodia; with alumina, such as the products “Tipaque TTO-55 (B)” and “Tipaque TTO-55 (A)” from Ishihara, and “UVT 14/4” from Kemira; with alumina and with aluminum stearate, such as the product “Microtitanium Dioxide MT 100 T, MT 100 TX, MT 100 Z or MT-01” from Tayca, the products “Solaveil CT-10 W” and “Solaveil CT 100” from Uniqema, and the product “Eusolex T-AVO” from Merck; with alumina and with aluminum laurate, such as the product “Microtitanium Dioxide MT 100 S” from Tayca; with iron oxide and with iron stearate, such as the product “Microtitanium Dioxide MT 100 F” from Tayca; with zinc oxide and with zinc stearate, such as the product “BR351” from Tayca; with silica and with alumina and treated with a silicone, such as the products “Microtitanium Dioxide MT 600 SAS”, “Microtitanium Dioxide MT 500 SAS”, and “Microtitanium Dioxide MT 100 SAS” from Tayca; with silica, with alumina and with aluminum stearate and treated with a silicone, such as the product “STT-30-DS” from Titan Kogyo; with silica and treated with a silicone, such as the product “UV-Titan X 195” from Kemira; with alumina and treated with a silicone, such as the products “Tipaque TTO-55 (S)” from Ishihara or “UV Titan M 262” from Kemira; with triethanolamine, such as the product “STT-65-S” from Titan Kogyo; with stearic acid, such as the product “Tipaque TTO-55 (C)” from Ishihara; or with sodium hexametaphosphate, such as the product “Microtitanium Dioxide MT 150 W” from Tayca. Other titanium oxide pigments treated with a silicone are, and in some embodiments TiO2 treated with octyltrimethylsilane and for which the mean size of the individual particles is from 25 and 40 nm, such as that marketed under the trademark “T 805” by Degussa Silices, TiO2 treated with a polydimethylsiloxane and for which the mean size of the individual particles is 21 nm, such as that marketed under the trademark “70250 Cardre UF TiO2Si3” by Cardre, and anatase/rutile TiO2 treated with a polydimethylhydrosiloxane and for which the mean size of the individual particles is 25 nm, such as that marketed under the trademark “Microtitanium Dioxide USP Grade Hydrophobic” by Color Techniques.

And in some embodiments, the following coated TiO2 can be used as the coated mineral UV filter: Stearic acid (and) Aluminum Hydroxide (and) TiO2, such as the product “MT-100 TV” from Tayca, with a mean primary particle diameter of 15 nm; Dimethicone (and) Stearic Acid (and) Aluminum Hydroxide (and) TiO2, such as the product “S A-TTO-S4” from Miyoshi Kasei, with a mean primary particle diameter of 15 nm; Silica (and) TiO2, such as the product “MT-100 WP” from Tayca, with a mean primary particle diameter of 15 nm; Dimethicone (and) Silica (and) Aluminum Hydroxide (and) TiO2, such as the product “MT-Y02” and “MT-Y-110 M3S” from Tayca, with a mean primary particle diameter of 10 nm; Dimethicone (and) Aluminum Hydroxide (and) TiO2, such as the product “SA-TTO-S3” from Miyoshi Kasei, with a mean primary particle diameter of 15 nm; Dimethicone (and) Alumina (and) TiO2, such as the product “UV TITAN MI 70” from Sachtleben, with a mean primary particle diameter of 15 nm;. and Silica (and) Aluminum Hydroxide (and) Alginic Acid (and) TiO2, such as the product “MT100 AQ” from Tayca, with a mean primary particle diameter of 15 nm. In terms of UV filtering ability, TiO2 coated with one or a combination of organic UV filter is more desirable. For example, Avobenzone (and) Stearic Acid (and) Aluminum Hydroxide (and) TiO2, such as the product “HXMT-100ZA” from Tayca, with a mean primary particle diameter of 15 nm, can be used.

e uncoated titanium oxide pigments are, for example, marketed by Tayca under the trademarks “Microtitanium Dioxide MT500B” or “Microtitanium Dioxide MT600B”, by Degussa under the trademark “P 25”, by Wacker under the trademark “Oxyde de titane transparent PW”, by Miyoshi Kasei under the trademark “UFTR”, by Tomen under the trademark “ITS” and by Tioxide under the trademark “Tioveil AQ”. The uncoated zinc oxide pigments are, for example: those marketed under the trademark “Z-cote” by Sunsmart; those marketed under the trademark “Nanox” by Elementis; and those marketed under the trademark “Nanogard WCD 2025” by Nanophase Technologies. The coated zinc oxide pigments are, for example: those marketed under the trademark “Oxide Zinc CS-5” by Toshiba (ZnO coated with polymethylhydrosiloxane); those marketed under the trademark “Nanogard Zinc Oxide FN” by Nanophase Technologies (as a 40% dispersion in Finsolv TN, C12-C15 alkyl benzoate); those marketed under the trademark “Daitopersion Zn-30” and “Daitopersion Zn-50” by Daito (dispersions in oxyethylenated polydimethylsiloxane/cyclopolymethylsiloxane comprising 30% or 50% of zinc nanooxides coated with silica and polymethylhydrosiloxane); those marketed under the trademark “NFD Ultrafine ZnO” by Daikin (ZnO coated with phosphate of perfiuoroalkyl and a copolymer based on perfluoroalkylethyl as a dispersion in cyclopentasiloxane); those marketed under the trademark “SPD-Z1” by Shin-Etsu (ZnO coated with a siliconegrafted acrylic polymer dispersed in cyclodimethylsiloxane); those marketed under the trademark “Escalol Z100” by ISP (alumina-treated ZnO dispersed in an ethylhexyl methoxycinnamate/PVP-hexadecene copolymer/methicone mixture); those marketed under the trademark “Fuji ZnO-SMS-10” by Fuji Pigment (ZnO coated with silica and polymethylsilsesquioxane); and those marketed under the trademark “Nanox Gel TN” by Elementis (ZnO dispersed at 55% in C12-C15 alkyl benzoate with hydroxystearic acid polycondensate). The uncoated cerium oxide pigments are marketed, for example, under the trademark “Colloidal Cerium Oxide” by Rhone-Poulenc.

The uncoated iron oxide pigments are, for example, marketed by Arnaud under the trademarks “Nanogard WCD 2002 (FE 45B)”, “Nanogard Iron FE 45 BL AQ”, “Nanogard FE 45R AQ”, and “Nanogard WCD 2006 (FE 45R)”, or by Mitsubishi under the trademark “TY-220”. The coated iron oxide pigments are, for example, marketed by Arnaud under the trademarks “Nanogard WCD 2008 (FE 45B FN)”, “Nanogard WCD 2009 (FE 45B 556)”, “Nanogard FE 45 BL 345”, and “Nanogard FE 45 BL”, or by BASF under the trademark “Oxyde de fer transparent”.

### Definitions and Terms

As used herein, the term “whitening efficiency” is defined as the color difference between untreated skin and skin cleansed with the coacervate cleansing system described.

As used herein, the phrases “and mixtures thereof,” “and a mixture thereof,” “and combinations thereof,” “and a combination thereof,” “or mixtures thereof,” “or a mixture thereof,” “or combinations thereof,” and “or a combination thereof,” are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

The transitional terms “comprising”, “consisting essentially of” and “consisting of”, when used in the appended claims, in original and amended form, define the claim scope with respect to what unrecited additional claim elements or steps, if any, are excluded from the scope of the claim(s). As used herein, the terms “comprising,” “having,” and “including” (or “comprise,” “have,” and “include”) are used in their open, non-limiting sense. The term “comprising” is intended to be inclusive or open-ended and does not exclude any additional, unrecited element, method, step, or material. The term “consisting of” excludes any element, step, or material other than those specified in the claim and, in the latter instance, impurities ordinary associated with the specified material(s). The term “consisting essentially of” limits the scope of a claim to the specified elements, steps, or material(s) and those that do not materially affect the basic and novel characteristic(s) of the claimed invention. All materials and methods described herein that embody the present invention can, in alternate embodiments, be more specifically defined by any of the transitional terms “comprising,” “consisting essentially of,” and “consisting of.”

In this application, the use of the singular includes the plural unless specifically stated otherwise. The singular forms “a,” “an,” “the,” and “at least one” are understood to encompass the plural as well as the singular unless the context clearly dictates otherwise, and these expressions, as well as the expression “one or more” which means “at least one,” are expressly intended to include the individual components as well as mixtures/combinations thereof.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. All ranges and amounts given herein are intended to include sub-ranges and amounts using any disclosed point as an end point. All numbers, amounts, ranges, etc., are intended to be modified by the term “about,” whether or not so expressly stated. Similarly, a range given of “about 3% to 7%” is intended to have the term “about” modifying both the 3% and the 7% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Likewise, all endpoints of ranges are understood to be individually disclosed, such that, for example, a range of 1:2 to 2:1 is understood to disclose a ratio of both 1:2 and 2:1.

“Active material” as used herein with respect to the percent amount of an ingredient or raw material, refers to 100% activity of the ingredient or raw material.

All amounts given herein are relative to the amount of active material, unless otherwise indicated.

All percentages, parts and ratios herein are based upon the total weight of the compositions of the present disclosure, unless otherwise indicated.

As used herein, the term “surfactants,” as well as any specifically-identified surfactants, includes salts of the surfactants even if not explicitly stated.

As used herein, the term “synthetic” means a material that is not of natural origin. The term “natural” and “naturally-sourced” and “nature-based” means a material of natural origin, such as derived from plants, which also cannot be subsequently chemically or physically modified. “Plant-based” means that the material came from a plant.

Unless otherwise expressly stated, it is in no way intended that any method set forth herein be construed as requiring that its steps be performed in a specific order. Accordingly, where a method claim does not expressly recite an order to be followed by its steps or it is not specifically stated in the claims or descriptions that the steps are to be limited to a specific order, it is no way intended that any particular order be inferred.

As used herein, the term “substantially free” or “essentially free” as used herein means the specific material may be present in small amounts that do not materially affect the basic and novel characteristics of the compositions according to the disclosure. For instance, there may be less than 2% by weight of a specific material added to a composition, based on the total weight of the compositions (provided that an amount of less than 2% by weight does not materially affect the basic and novel characteristics of the compositions according to the disclosure. Similarly, the compositions may include less than 2%, less than 1.5%, less than 1%, less than 0.5%, less than 0.1%, less than 0.05%, or less than 0.01%, or none of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure. The term “substantially free” or “essentially free” as used herein may also mean that the specific material is not added to the composition but may still be present in a raw material that is included in the composition.

### EXAMPLES

The following examples are intended to be non-limiting and explanatory in nature only. In the Examples, amounts are expressed in percentage by weight (wt %) of active materials, relative to the total weight of the composition.

### Example 1—Coacervate Examples

In order to demonstrate the coacervate phenomenon, the following compositions A-D were prepared.

TABLE 2
Examples of coacervate systems

| | Coacervate Compositions | | | |
|---|---|---|---|---|
| INCI Name | A | B | C | D |
| Cocamidopropyl | 5% | 5% | 5% | |
| hydroxysultaine | | | | |
| Coco Betaine | | | | 5% |
| Rhamnolipid | | | 4% | 4% |
| Sophorolipid | 4% | 4% | | |
| Chitosan | | 0.1% | | 0.1% |
| Polylysine | 0.2% | | 0.1% | |
| Water | 90.8% | 90.9% | 90.9% | 90.9% |
| pH | 6.2 | 5.2 | 5.2 | 4.85 |
| Phase behavior | hazy | hazy | hazy | hazy |
| after 10x dilution | | | | |

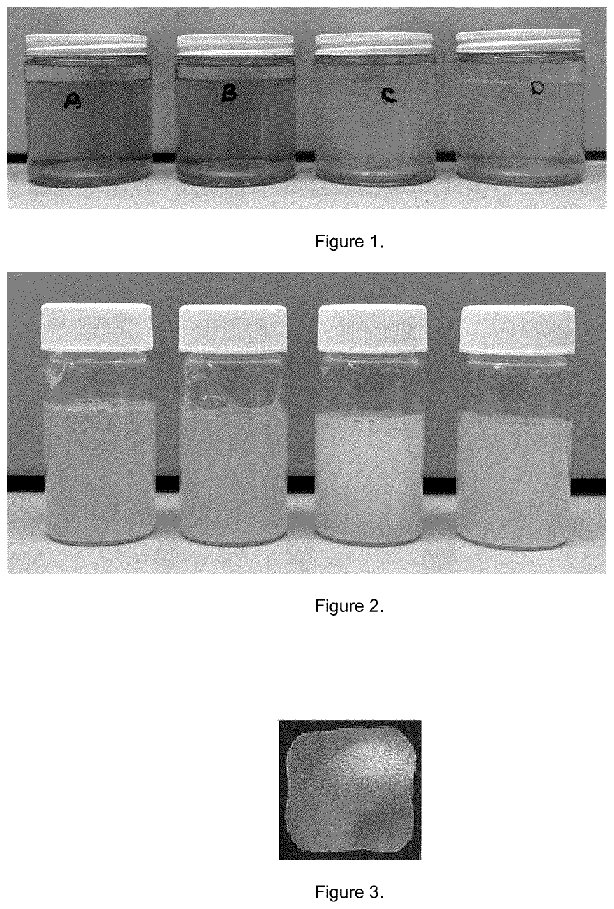

Each of compositions (A-D) were clear upon initial preparation as shown in FIG. 1. After dilution 10 times with water, the compositions appeared hazy, as shown in FIG. 2. Each of compositions A-D demonstrated the coacervate phenomenon by going through the phase transition during dilution with water.

### Example 2—Coacervate System with Sophorolipid

In order to demonstrate the coacervate phenomenon using sophorolipid, the following compositions (3-7) and comparative compositions (1,2,8, and 9) were prepared.

TABLE 3
Compositions with Cocamidopropyl Hydroxysultaine,
Sophorolipid and Chitosan - Behavior After Dilution

| | | | | | | Phase Behavior |
|---|---|---|---|---|---|---|
| | A | B | Chitosan | | | after dilution |
| Sample | % | % | % | Water | pH | 2-10x |
| 1 | 1% | 9% | 0.1% | 89.9% | 4.5-6.5 | Solution not clear |
| 2 | 2% | 8% | 0.1% | 89.9% | 4.5-6.5 | Solution not clear |
| 3 | 3% | 7% | 0.1% | 89.9% | 4.5-6.5 | Cloudy/precipitates |
| | | | | | | when diluted |
| 4 | 4% | 6% | 0.1% | 89.9% | 4.5-6.5 | Cloudy/precipitates |
| | | | | | | when diluted |
| 5 | 5% | 5% | 0.1% | 89.9% | 4.5-6.5 | Cloudy/precipitates |
| | | | | | | when diluted |
| 6 | 6% | 4% | 0.1% | 89.9% | 4.5-6.5 | Cloudy/precipitates |
| | | | | | | when diluted |
| 7 | 7% | 3% | 0.1% | 89.9% | 4.5-6.5 | Cloudy/precipitates |
| | | | | | | when diluted |
| 8 | 8% | 2% | 0.1% | 89.9% | 4.5-6.5 | No coacervate |
| 9 | 9% | 1% | 0.1% | 89.9% | 4.5-6.5 | No coacervate |

A = Cocamidopropyl Hydroxysultaine;
B = Sophorolipid

Compositions 1-9 were prepared, and then diluted 10 times with water. Samples 3-7 demonstrated the coacervate phenomenon and turned cloudy after dilution with water. Samples 1,2,8, and 9 did not demonstrate coacervate, and did not turn cloudy after dilution. Samples with sophorolipid concentrations between 3 and 7%, and cocamidopropyl hydroxysultaine concentrations between 3 and 7% demonstrated coacervate.

### Example 3—Coacervate System with Rhamnolipid

In order to demonstrate the coacervate phenomenon could be achieved across a range of concentrations, the following compositions containing varying amounts of cocamidopropyl hydroxysultaine, rhamnolipid, and polylysine were prepared.

TABLE 4
Compositions with Cocamidopropyl Hydroxysultaine, Rhamnolipid
and Polylysine - Behavior After Dilution

| | A | C | Polylysine | | | |
|---|---|---|---|---|---|---|
| Sample | % | % | % | Water | pH | Phase Behavior |
| 10 | 1% | 9% | 0.1% | 89.9% | 4.5-7.5 | No coacervate |
| 11 | 2% | 8% | 0.1% | 89.9% | 4.5-7.5 | No coacervate |
| 12 | 3% | 7% | 0.1% | 89.9% | 4.5-7.5 | Cloudy/precipitates |
| | | | | | | when diluted |
| 13 | 4% | 6% | 0.1% | 89.9% | 4.5-7.5 | Cloudy/precipitates |
| | | | | | | when diluted |
| 14 | 5% | 5% | 0.1% | 89.9% | 4.5-7.5 | Cloudy/precipitates |
| | | | | | | when diluted |
| 15 | 6% | 4% | 0.1% | 89.9% | 4.5-7.5 | Cloudy/precipitates |
| | | | | | | when diluted |
| 16 | 7% | 3% | 0.1% | 89.9% | 4.5-7.5 | Cloudy/precipitates |
| | | | | | | when diluted |
| 17 | 8% | 2% | 0.1% | 89.9% | 4.5-7.5 | Cloudy/precipitates |
| | | | | | | when diluted |
| 18 | 9% | 1% | 0.1% | 89.9% | 4.5-7.5 | No Coacervate |

A = Cocamidopropyl Hydroxysultaine;
C = Rhamnolipid

Compositions 10-18 were prepared, and then diluted 10 times with water. Samples 12-17 demonstrated the coacervate phenomenon and turned cloudy after dilution with water. Samples 10,11, and 18 did not demonstrate coacervate, and did not turn cloudy after dilution. Samples with rhamnolipid concentrations between 2 and 7%, and cocamidopropyl hydroxysultaine concentrations between 3 and 8% demonstrated coacervate.

### Example 4—Comparative Systems with Mineral Particles Examples

In order to demonstrate the deposition of mineral particles according to the disclosure, the following inventive coacervate composition (19) and comparative non-coacervate compositions (20&21) were prepared.

TABLE 5
Inventive and Comparative Formulations

| | 19 - | 20 - | 21 - |
|---|---|---|---|
| | Inventive | Comparative | Comparative |
| INCI Name | Formula | Formula | Formula |
| Cocamidopropyl | 5% | 5% | |
| Hydroxysultaine | | | |
| Rhamnolipid | 4% | 4% | |
| Titanium Dioxide | 2.5% | 2.5% | 2.5% |
| Polylysine | 0.1% | | |
| Water | 88.4% | 88.5% | 47.6% |
| Other Surfactants | | | 22% |
| Active Compounds | | | 6.2% |
| Fragrance | | | 0.9% |
| Polymers | | | 12.1% |
| Preservatives | | | 0.7% |
| Other Solvents | | | 8% |
| pH | 5.2 | 5.2 | 8.6 |

### Example 5—Whitening Efficiency

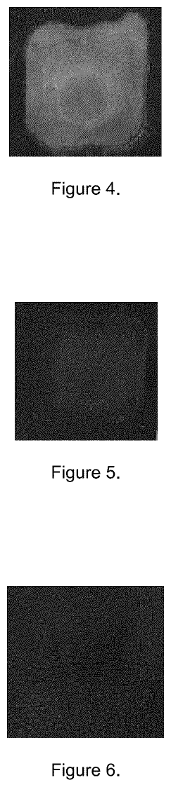

In order to demonstrate the deposition of TiO2 left after washing, samples 19-21 were prepared. 0.2 g of each sample were placed on a black substrate and spread evenly across a 1 inch area, as shown in FIG. 3. The samples were then rinsed with water and examined for residual TiO2 as shown in FIGS. 4-6. The whitening efficiency was measured for each sample, and the results are shown in Table 6 below.

Method of determination of whitening efficiency was measured by Datacolor 600.

Baseline: completely clean substrate, without cleanser applied.

Sample before rinse off: substrate with cleanser applied, and before removal performed.

Sample after rinse off: substrate with cleanser applied, and after removal performed.

The equation below was used to calculate the whitening efficacy.

$$Whitening efficacy = \frac{{De}_{sample after rinse off} - {De}_{baseline}}{{De}_{sample before rinse off} - {De}_{baseline}}$$

Referring to table 5, the coacervate composition (19) was tested and compared with the non-coacervate compositions (20&21). The results are shown below in Table 6.

TABLE 6
Whitening Efficiency

| Formula | Whitening Efficiency % |
|---|---|
| 19 | 64.7% |
| 20 | 6.8% |
| 21 | 3.5% |

The coacervate composition (19) left a visible layer of TiO2 as shown in FIG. 4, and had the highest whitening efficiency of 64.7%. The coacervate composition (19) showed much higher whitening efficiency as compared to the non-coacervate compositions (20&21) shown in FIGS. 5 and 6. The coacervate composition (19) showed an increase in deposition of TiO2 compared to the non-coacervate compositions (20&21).

1. A coacervate cleansing system, comprising:
(a) at least one amphoteric surfactant;
(b) at least one glycolipid;
(c) at least one cationic polymer from about 0.05% to about 2%;
(d) at least one mineral particle; and
(e) at least one cosmetically acceptable solvent;
wherein the cleansing system is essentially free of phenoxyethanol and
wherein the pH of the composition is between 4 and 8, and
wherein the weight ratio of a to b is from about 0.4 to about 4, and
wherein the total concentration of a and b combined is about 10 to about 20% of the total weight of the composition.

2. The composition of claim 1, wherein the at least one amphoteric surfactant is chosen from cocamidopropyl hydroxysultaine, cocamidopropyl betaine, coco betaine, sodium lauroamphoacetate, disodium cocoamphodiacetate or a mixture thereof.

3. The composition of claim 1, wherein the at least one glycolipid is chosen from rhamnolipids, sophorolipids, glucolipids, trehalolipids, cellobiose lipids, mannosylerythritol lipid, or a mixture thereof.

4. The composition of claim 1, wherein the at least one cationic polymer is chosen from chitosan, chitosan oligosaccharide, chitin, cyclodextrin, cationic gelatin, cationic dextran, cationic cellulose, polylysine, polyornithine, histone, collagen, chitosan-cysteine, chitosan-thiobutylamidine, chitosan-thioglycolic acid, or a mixture thereof.

5. The composition of claim 1, wherein the at least one mineral particle is chosen from titanium dioxide, zinc oxide, and mixtures thereof.

6. The composition of claim 1, further comprising at least one additional cosmetic ingredient.

7. The composition of claim 1, wherein the at least one cationic polymer is preferably chosen from a nature-based polymer.

8. The composition of claim 2, wherein the at least one amphoteric surfactant is chosen from cocamidopropyl hydroxysultaine, coco-betaine, or a mixture thereof.

9. The composition of claim 3, wherein the at least one glycolipid is chosen from sophorolipid, rhamnolipid, or a mixture thereof.

10. The composition of claim 4, wherein the at least one cationic polymer is chosen from chitosan, polylysine, or a mixture thereof.

11. The composition of claim 2, wherein the concentration of amphoteric surfactant is from about 3% to about 8%.

12. The composition of claim 3, wherein the concentration of glycolipid is from about 2% to about 7%.

13. The composition of claim 4, wherein the concentration of cationic polymer is from about 0.1 to about 0.2%.

14. The composition of claim 5, wherein the concentration of mineral particle is from about 1% to about 10%.

15. The composition of claim 1, wherein the pH is from 4.5 to 7.5.

16. A coacervate cleansing system, comprising:
(a) at least one amphoteric surfactant selected from the group consisting of cocamidopropyl hydroxysultaine and coco betaine from about 3% to about 8% by weight, relative to the total weight of the composition;
(b) at least one glycolipid selected from the group consisting of rhamnolipid and sophorolipid from about 2% to about 7% by weight, relative to the total weight of the composition;
(c) at least one cationic polymer selected from the group consisting of chitosan and polylysine from about 0.1% to about 0.2%;
(d) at least one particle filter selected from the group consisting of titanium dioxide and zinc oxide from about 1% to about 10%; and
(e) Water;
wherein the cleansing system is essentially free of phenoxyethanol, and
wherein the pH of the composition is between 4 and 8, and
wherein the weight ratio of a to b is about 0.4 to about 4.

17. A method for depositing at least one mineral particle to the skin during cleansing comprising (i) applying to the skin of a subject a coacervate cleansing system, comprising:
(a) at least one amphoteric surfactant;
(b) at least one glycolipid;
(c) at least one cationic polymer from about 0.05% to about 2%;
(d) at least one mineral particle; and
(e) at least one cosmetically acceptable solvent
wherein the cleansing system is essentially free of phenoxyethanol, and
wherein the pH of the composition is between about 4 and about 8, and
wherein the weight ratio of a to b is about 0.4 to about 4, and
wherein the total concentration of a and b combined is about 10% to about 20% of the total weight of the composition, and
(ii) removing the cleansing system from the skin.

18. The method of claim 17, wherein the method further comprises a step of wetting the skin of a subject before applying a coacervate cleansing system.
